(19) United States
(12) Patent Application Publication
(10) Pub. No.: US 2023/0020346 A1
(43) Pub. Date: Jan. 19, 2023

(54) SCENE ADAPTIVE ENDOSCOPIC HYPERSPECTRAL IMAGING SYSTEM

(71) Applicant: Cilag GmbH International, Zug (CH)

(72) Inventors: Tarik Yardibi, Wayland, MA (US); Emir Osmanagic, Norwell, MA (US); Patrick J. Treado, Pittsburgh, PA (US); Jeffrey Beckstead, Valencia, PA (US); Matthew P. Nelson, Harrison City, PA (US); Alyssa Zrimsek, Pittsburgh, PA (US); Nathaniel Gomer, Sewickley, PA (US)

(21) Appl. No.: 17/375,281

(22) Filed: Jul. 14, 2021

(51) Int. Cl. A61B 1/06; A61B 5/00; A61B 5/107; A61B 1/00

(52) U.S. Cl. CPC A61B 1/0638; A61B 5/489; A61B 5/4893; A61B 5/4222; A61B 5/1076; A61B 1/063; A61B 1/0005

(57) ABSTRACT

A method of operating a surgical visualization system includes illuminating an anatomical field of a patient using a waveform transmitted by an emitter. The method also includes capturing an image of the anatomical field based on the waveform using a receiver. The emitter and the receiver are configured for multispectral imaging or hyperspectral imaging. The method also includes determining an adjustment to at one operating parameter of the surgical visualization system based on at least one environmental scene parameter. The method also includes automatically implementing the adjustment to the at least one operating parameter to aid in identification of at least one anatomical structure in the anatomical field.

### BACKGROUND

Surgical systems may incorporate an imaging system, which may allow the clinician(s) to view the surgical site and/or one or more portions thereof on one or more displays such as a monitor. The display(s) may be local and/or remote to a surgical theater. An imaging system may include a scope with a camera that views the surgical site and transmits the view to a display that is viewable by the clinician. Scopes include, but are not limited to, laparoscopes, robotic laparoscopes, arthroscopes, angioscopes, bronchoscopes, choledochoscopes, colonoscopes, cytoscopes, duodenoscopes, enteroscopes, esophagogastro-duodenoscopes (gastroscopes), endoscopes, laryngoscopes, nasopharyngo-neproscopes, sigmoidoscopes, thoracoscopes, ureteroscopes, and exoscopes. Imaging systems may be limited by the information that they are able to recognize and/or convey to the clinician(s). For example, certain concealed structures, physical contours, and/or dimensions within a three-dimensional space may be unrecognizable intraoperatively by certain imaging systems. Additionally, certain imaging systems may be incapable of communicating and/or conveying certain information to the clinician(s) intraoperatively.

Examples of surgical imaging systems are disclosed in U.S. Pat. Pub. No. 2020/0015925, entitled “Combination Emitter and Camera Assembly,” published Jan. 16, 2020; U.S. Pat. Pub. No. 2020/0015899, entitled “Surgical Visualization with Proximity Tracking Features,” published Jan. 16, 2020; U.S. Pat. Pub. No. 2020/0015924, entitled “Robotic Light Projection Tools,” published Jan. 16, 2020; and U.S. Pat. Pub. No. 2020/0015898, entitled “Surgical Visualization Feedback System,” published Jan. 16, 2020. The disclosure of each of the above-cited U.S. patents and patent applications is incorporated by reference herein in its entirety.

While various kinds of surgical instruments and systems have been made and used, it is believed that no one prior to the inventor(s) has made or used the invention described in the appended claims.

### BRIEF DESCRIPTION OF THE DRAWINGS

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 depicts a schematic view of an exemplary surgical visualization system including an imaging device and a surgical device;

FIG. 2 depicts a schematic diagram of an exemplary control system that may be used with the surgical visualization system of FIG. 1;

FIG. 3 depicts a schematic diagram of another exemplary control system that may be used with the surgical visualization system of FIG. 1;

FIG. 4 depicts exemplary hyperspectral identifying signatures to differentiate anatomy from obscurants, and more particularly depicts a graphical representation of a ureter signature versus obscurants;

FIG. 5 depicts exemplary hyperspectral identifying signatures to differentiate anatomy from obscurants, and more particularly depicts a graphical representation of an artery signature versus obscurants;

FIG. 6 depicts exemplary hyperspectral identifying signatures to differentiate anatomy from obscurants, and more particularly depicts a graphical representation of a nerve signature versus obscurants;

FIG. 7A depicts a schematic view of an exemplary emitter assembly that may be incorporated into the surgical visualization system of FIG. 1, the emitter assembly including a single electromagnetic radiation (EMR) source, showing the emitter assembly in a first state;

FIG. 7B depicts a schematic view of the emitter assembly of FIG. 7A, showing the emitter assembly in a second state;

FIG. 7C depicts a schematic view of the emitter assembly of FIG. 7A, showing the emitter assembly in a third state;

FIG. 8 depicts a schematic view of another exemplary surgical visualization system;

FIG. 9 depicts a flow diagram of an exemplary method of automatically adjusting one or more environmental scene parameters based on adjustments to one or more operating parameters;

FIG. 10 depicts exemplary correlations between environmental scene parameters and operating parameters;

FIG. 11 depicts a flow diagram of an exemplary method of automatically adjusting one or more operating parameters based on pre-operative information;

FIG. 12A depicts a graph of an exemplary plot of the relationship between standard and high signal to noise ratios relative to obscuration depth;

FIG. 12B depicts a table of the relationship between the standard and high signal to noise ratios relative to the obscuration depth of FIG. 12A;

FIG. 13 depicts an exemplary anatomical field that includes critical and background structures;

FIG. 14 depicts an image of the anatomical field of FIG. 13, but with the critical and background structures displayed using masks when the image is captured at a first emitter power; and

FIG. 15 depicts an image of the anatomical field of FIG. 13, but with the critical and background structures using masks when the image is captured at a second emitter power.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

For clarity of disclosure, the terms “proximal” and “distal” are defined herein relative to a surgeon, or other operator, grasping a surgical device. The term “proximal” refers to the position of an element arranged closer to the surgeon, and the term “distal” refers to the position of an element arranged further away from the surgeon. Moreover, to the extent that spatial terms such as “top,” “bottom,” “upper,” “lower,” “vertical,” “horizontal,” or the like are used herein with reference to the drawings, it will be appreciated that such terms are used for exemplary description purposes only and are not intended to be limiting or absolute. In that regard, it will be understood that surgical instruments such as those disclosed herein may be used in a variety of orientations and positions not limited to those shown and described herein.

Furthermore, the terms “about,” “approximately,” and the like as used herein in connection with any numerical values or ranges of values are intended to encompass the exact value(s) referenced as well as a suitable tolerance that enables the referenced feature or combination of features to function for the intended purpose(s) described herein.

Similarly, the phrase “based on” should be understood as referring to a relationship in which one thing is determined at least in part by what it is specified as being “based on.” This includes, but is not limited to, relationships where one thing is exclusively determined by another, which relationships may be referred to using the phrase “exclusively based on.”

I. Exemplary Surgical Visualization System

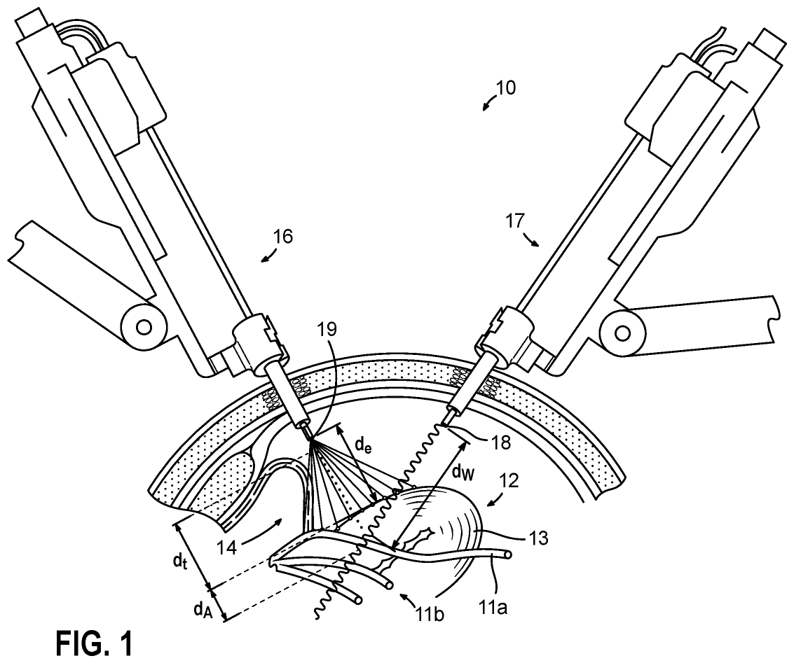

FIG. 1 depicts a schematic view of a surgical visualization system (10) according to at least one aspect of the present disclosure. The surgical visualization system (10) may create a visual representation of a critical structure (11a, 11b) within an anatomical field. The surgical visualization system (10) may be used for clinical analysis and/or medical intervention, for example. In certain instances, the surgical visualization system (10) may be used intraoperatively to provide real-time, or near real-time, information to the clinician regarding proximity data, dimensions, and/or distances during a surgical procedure. The surgical visualization system (10) is configured for intraoperative identification of critical structure(s) and/or to facilitate the avoidance of critical structure(s) (11a, 11b) by a surgical device. For example, by identifying critical structures (11a, 11b), a clinician may avoid maneuvering a surgical device into a critical structure (11a, 11b) and/or a region in a predefined proximity of a critical structure (11a, 11b) during a surgical procedure. The clinician may avoid dissection of and/or near a vein, artery, nerve, and/or vessel, for example, identified as a critical structure (11a, 11b), for example. In various instances, critical structure(s) (11a, 11b) may be determined on a patient-by-patient and/or a procedure-by-procedure basis.

Critical structures (11a, 11b) may be any anatomical structures of interest. For example, a critical structure (11a, 11b) may be a ureter, an artery such as a superior mesenteric artery, a vein such as a portal vein, a nerve such as a phrenic nerve, and/or a sub-surface tumor or cyst, among other anatomical structures. In other instances, a critical structure (11a, 11b) may be any foreign structure in the anatomical field, such as a surgical device, surgical fastener, clip, tack, bougie, band, and/or plate, for example. In one aspect, a critical structure (11a, 11b) may be embedded in tissue. Stated differently, a critical structure (11a, 11b) may be positioned below a surface of the tissue. In such instances, the tissue conceals the critical structure (11a, 11b) from the clinician's view. A critical structure (11a, 11b) may also be obscured from the view of an imaging device by the tissue. The tissue may be fat, connective tissue, adhesions, and/or organs, for example. In other instances, a critical structure (11a, 11b) may be partially obscured from view. A surgical visualization system (10) is shown being utilized intraoperatively to identify and facilitate avoidance of certain critical structures, such as a ureter (11a) and vessels (11b) in an organ (12) (the uterus in this example), that are not visible on a surface (13) of the organ (12).

A. Overview of Exemplary Surgical Visualization System

With continuing reference to FIG. 1, the surgical visualization system (10) incorporates tissue identification and geometric surface mapping in combination with a distance sensor system (14). In combination, these features of the surgical visualization system (10) may determine a position of a critical structure (11a, 11b) within the anatomical field and/or the proximity of a surgical device (16) to the surface (13) of the visible tissue and/or to a critical structure (11a, 11b). The surgical device (16) may include an end effector having opposing jaws (not shown) and/or other structures extending from the distal end of the shaft of the surgical device (16). The surgical device (16) may be any suitable surgical device such as, for example, a dissector, a stapler, a grasper, a clip applier, a monopolar RF electrosurgical instrument, a bipolar RF electrosurgical instrument, and/or an ultrasonic instrument. As described herein, a surgical visualization system (10) may be configured to achieve identification of one or more critical structures (11a, 11b) and/or the proximity of a surgical device (16) to critical structure(s) (11a, 11b).

The depicted surgical visualization system (10) includes an imaging system that includes an imaging device (17), such as a camera of a scope, for example, that is configured to provide real-time views of the surgical site. In various instances, an imaging device (17) includes a spectral camera (e.g., a hyperspectral camera, multispectral camera, a fluorescence detecting camera, or selective spectral camera), which is configured to detect reflected or emitted spectral waveforms and generate a spectral cube of images based on the molecular response to the different wavelengths. Views from the imaging device (17) may be provided to a clinician; and, in various aspects of the present disclosure, may be augmented with additional information based on the tissue identification, landscape mapping, and input from a distance sensor system (14). In such instances, a surgical visualization system (10) includes a plurality of subsystems—an imaging subsystem, a surface mapping subsystem, a tissue identification subsystem, and/or a distance determining subsystem. These subsystems may cooperate to intraoperatively provide advanced data synthesis and integrated information to the clinician(s).

The imaging device (17) of the present example includes an emitter (18), which is configured to emit spectral light in a plurality of wavelengths to obtain a spectral image of hidden structures, for example. The imaging device (17) may also include a three-dimensional camera and associated electronic processing circuits in various instances. In one aspect, the emitter (18) is an optical waveform emitter that is configured to emit electromagnetic radiation (e.g., near-infrared radiation (NIR) photons) that may penetrate the surface (13) of a tissue (12) and reach critical structure(s) (11a, 11b). The imaging device (17) and optical waveform emitter (18) thereon may be positionable by a robotic arm or a surgeon manually operating the imaging device. A corresponding waveform sensor (e.g., an image sensor, spectrometer, or vibrational sensor, etc.) on the imaging device (17) may be configured to detect the effect of the electromagnetic radiation received by the waveform sensor.

The wavelengths of the electromagnetic radiation emitted by the optical waveform emitter (18) may be configured to enable the identification of the type of anatomical and/or physical structure, such as critical structure(s) (11a, 11b). The identification of critical structure(s) (11a, 11b) may be accomplished through spectral analysis, photo-acoustics, fluorescence detection, and/or ultrasound, for example. In one aspect, the wavelengths of the electromagnetic radiation may be variable. The waveform sensor and optical waveform emitter (18) may be inclusive of a multispectral imaging system and/or a selective spectral imaging system, for example. In other instances, the waveform sensor and optical waveform emitter (18) may be inclusive of a photoacoustic imaging system, for example. In other instances, an optical waveform emitter (18) may be positioned on a separate surgical device from the imaging device (17). By way of example only, the imaging device (17) may provide hyperspectral imaging in accordance with at least some of the teachings of U.S. Pat. No. 9,274,047, entitled “System and Method for Gross Anatomic Pathology Using Hyperspectral Imaging,” issued Mar. 1, 2016, the disclosure of which is incorporated by reference herein in its entirety.

The depicted surgical visualization system (10) also includes an emitter (19), which is configured to emit a pattern of light, such as stripes, grid lines, and/or dots, to enable the determination of the topography or landscape of a surface (13). For example, projected light arrays may be used for three-dimensional scanning and registration on a surface (13). The projected light arrays may be emitted from an emitter (19) located on a surgical device (16) and/or an imaging device (17), for example. In one aspect, the projected light array is employed to determine the shape defined by the surface (13) of the tissue (12) and/or the motion of the surface (13) intraoperatively. An imaging device (17) is configured to detect the projected light arrays reflected from the surface (13) to determine the topography of the surface (13) and various distances with respect to the surface (13). By way of further example only, a visualization system (10) may utilize patterned light in accordance with at least some of the teachings of U.S. Pat. Pub. No. 2017/0055819, entitled “Set Comprising a Surgical Instrument,” published Mar. 2, 2017, the disclosure of which is incorporated by reference herein in its entirety; and/or U.S. Pat. Pub. No. 2017/0251900, entitled “Depiction System,” published Sep. 7, 2017, the disclosure of which is incorporated by reference herein in its entirety.

The depicted surgical visualization system (10) also includes a distance sensor system (14) configured to determine one or more distances at the surgical site. In one aspect, the distance sensor system (14) may include a time-of-flight distance sensor system that includes an emitter, such as the structured light emitter (19); and a receiver (not shown), which may be positioned on the surgical device (16). In other instances, the time-of-flight emitter may be separate from the structured light emitter (19). In one general aspect, the emitter portion of the time-of-flight distance sensor system (14) may include a laser source and the receiver portion of the time-of-flight distance sensor system (14) may include a matching sensor. A time-of-flight distance sensor system (14) may detect the “time of flight,” or how long the laser light emitted by the structured light emitter (19) has taken to bounce back to the sensor portion of the receiver. Use of a very narrow light source in a structured light emitter (19) may enable a distance sensor system (14) to determine the distance to the surface (13) of the tissue (12) directly in front of the distance sensor system (14).

Referring still to FIG. 1, a distance sensor system (14) may be employed to determine an emitter-to-tissue distance (dₑ) from a structured light emitter (19) to the surface (13) of the tissue (12). A device-to-tissue distance (dₜ) from the distal end of the surgical device (16) to the surface (13) of the tissue (12) may be obtainable from the known position of the emitter (19) on the shaft of the surgical device (16) relative to the distal end of the surgical device (16). In other words, when the distance between the emitter (19) and the distal end of the surgical device (16) is known, the device-to-tissue distance (dₜ) may be determined from the emitter-to-tissue distance (dₑ). In certain instances, the shaft of a surgical device (16) may include one or more articulation joints; and may be articulatable with respect to the emitter (19) and the jaws. The articulation configuration may include a multi-joint vertebrae-like structure, for example. In certain instances, a three-dimensional camera may be utilized to triangulate one or more distances to the surface (13).

As described above, a surgical visualization system (10) may be configured to determine the emitter-to-tissue distance (dₑ) from an emitter (19) on a surgical device (16) to the surface (13) of a uterus (12) via structured light. The surgical visualization system (10) is configured to extrapolate a device-to-tissue distance (dₜ) from the surgical device (16) to the surface (13) of the uterus (12) based on emitter-to-tissue distance (dₑ). The surgical visualization system (10) is also configured to determine a tissue-to-ureter distance (d_{A}) from a ureter (11a) to the surface (13) and a camera-to-ureter distance (d_{w}), from the imaging device (17) to the ureter (11a). Surgical visualization system (10) may determine the camera-to-ureter distance (d_{w}), with spectral imaging and time-of-flight sensors, for example. In various instances, a surgical visualization system (10) may determine (e.g., triangulate) a tissue-to-ureter distance (d_{A}) (or depth) based on other distances and/or the surface mapping logic described herein.

B. First Exemplary Control System

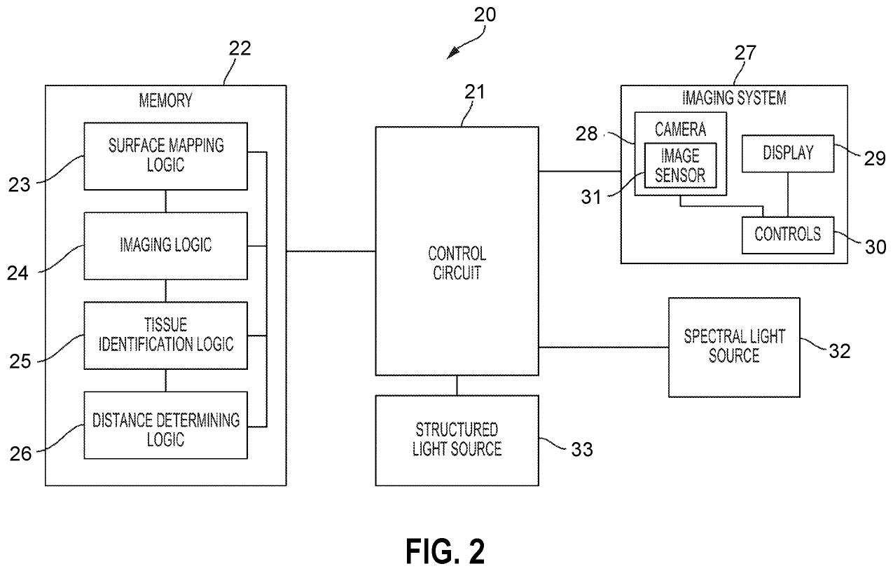

FIG. 2 is a schematic diagram of a control system (20), which may be utilized with a surgical visualization system (10). The depicted control system (20) includes a control circuit (21) in signal communication with a memory (22). The memory (22) stores instructions executable by the control circuit (21) to determine and/or recognize critical structures (e.g., critical structures (11a, 11b) depicted in FIG. 1), determine and/or compute one or more distances and/or three-dimensional digital representations, and to communicate certain information to one or more clinicians. For example, a memory (22) stores surface mapping logic (23), imaging logic (24), tissue identification logic (25), or distance determining logic (26) or any combinations of logic (23, 24, 25, 26). The control system (20) also includes an imaging system (27) having one or more cameras (28) (like the imaging device (17) depicted in FIG. 1), one or more displays (29), one or more controls (30) or any combinations of these elements. The one or more cameras (28) may include one or more image sensors (31) to receive signals from various light sources emitting light at various visible and invisible spectra (e.g., visible light, spectral imagers, three-dimensional lens, among others). The display (29) may include one or more screens or monitors for depicting real, virtual, and/or virtually-augmented images and/or information to one or more clinicians.

In various aspects, a main component of a camera (28) includes an image sensor (31). An image sensor (31) may include a Charge-Coupled Device (CCD) sensor, a Complementary Metal Oxide Semiconductor (CMOS) sensor, a short-wave infrared (SWIR) sensor, a hybrid CCD/CMOS architecture (sCMOS) sensor, and/or any other suitable kind(s) of technology. An image sensor (31) may also include any suitable number of chips.

The depicted control system (20) also includes a spectral light source (32) and a structured light source (33). In certain instances, a single source may be pulsed to emit wavelengths of light in the spectral light source (32) range and wavelengths of light in the structured light source (33) range. Alternatively, a single light source may be pulsed to provide light in the invisible spectrum (e.g., infrared spectral light) and wavelengths of light on the visible spectrum. A spectral light source (32) may include a hyperspectral light source, a multispectral light source, a fluorescence excitation light source, and/or a selective spectral light source, for example. In various instances, tissue identification logic (25) may identify critical structure(s) via data from a spectral light source (32) received by the image sensor (31) portion of a camera (28). Surface mapping logic (23) may determine the surface contours of the visible tissue based on reflected structured light. With time-of-flight measurements, distance determining logic (26) may determine one or more distance(s) to the visible tissue and/or critical structure(s) (11a, 11b). One or more outputs from surface mapping logic (23), tissue identification logic (25), and distance determining logic (26), may be provided to imaging logic (24), and combined, blended, and/or overlaid to be conveyed to a clinician via the display (29) of the imaging system (27).

C. Second Exemplary Control System

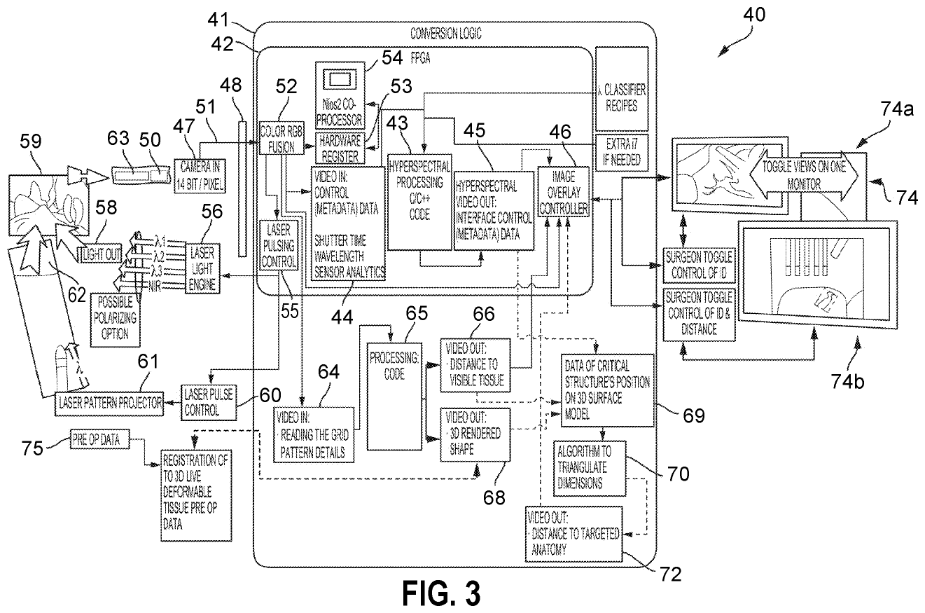

FIG. 3 depicts a schematic of another control system (40) for a surgical visualization system, such as the surgical visualization system (10) depicted in FIG. 1, for example. This control system (40) is a conversion system that integrates spectral signature tissue identification and structured light tissue positioning to identify critical structures, especially when those structures are obscured by other tissue, such as fat, connective tissue, blood, and/or other organs, for example. Such technology could also be useful for detecting tissue variability, such as differentiating tumors and/or non-healthy tissue from healthy tissue within an organ.

The control system (40) depicted in FIG. 3 is configured for implementing a hyperspectral or fluorescence imaging and visualization system in which a molecular response is utilized to detect and identify anatomy in a surgical field of view. This control system (40) includes a conversion logic circuit (41) to convert tissue data to surgeon usable information. For example, the variable reflectance based on wavelengths with respect to obscuring material may be utilized to identify a critical structure in the anatomy. Moreover, this control system (40) combines the identified spectral signature and the structured light data in an image. For example, this control system (40) may be employed to create a three-dimensional data set for surgical use in a system with augmentation image overlays. Techniques may be employed both intraoperatively and preoperatively using additional visual information. In various instances, this control system (40) is configured to provide warnings to a clinician when in the proximity of one or more critical structures. Various algorithms may be employed to guide robotic automation and semi-automated approaches based on the surgical procedure and proximity to the critical structure(s).

The control system (40) depicted in FIG. 3 is configured to detect the critical structure(s) and provide an image overlay of the critical structure and measure the distance to the surface of the visible tissue and the distance to the embedded/buried critical structure(s). In other instances, this control system (40) may measure the distance to the surface of the visible tissue or detect the critical structure(s) and provide an image overlay of the critical structure.

The control system (40) depicted in FIG. 3 includes a spectral control circuit (42). The spectral control circuit (42) includes a processor (43) to receive video input signals from a video input processor (44). The processor (43) is configured to process the video input signal from the video input processor (44) and provide a video output signal to a video output processor (45), which includes a hyperspectral video-out of interface control (metadata) data, for example. The video output processor (45) provides the video output signal to an image overlay controller (46).

The video input processor (44) is coupled to a camera (47) at the patient side via a patient isolation circuit (48). As previously discussed, the camera (47) includes a solid state image sensor (50). The camera (47) receives intraoperative images through optics (63) and the image sensor (50). An isolated camera output signal (51) is provided to a color RGB fusion circuit (52), which employs a hardware register (53) and a Nios2 co-processor (54) to process the camera output signal (51). A color RGB fusion output signal is provided to the video input processor (44) and a laser pulsing control circuit (55).

The laser pulsing control circuit (55) controls a light engine (56). In some versions, light engine (56) includes any one or more of lasers, LEDs, incandescent sources, and/or interface electronics configured to illuminate the patient's body habitus with a chosen light source for imaging by a camera and/or analysis by a processor. The light engine (56) outputs light in a plurality of wavelengths (λ1, λ2, λ3 . . . λn) including near infrared (NIR) and broadband white light. The light output (58) from the light engine (56) illuminates targeted anatomy in an intraoperative surgical site (59). The laser pulsing control circuit (55) also controls a laser pulse controller (60) for a laser pattern projector (61) that projects a laser light pattern (62), such as a grid or pattern of lines and/or dots, at a predetermined wavelength (λ2) on the operative tissue or organ at the surgical site (59). The camera (47) receives the patterned light as well as the reflected or emitted light output through camera optics (63). The image sensor (50) converts the received light into a digital signal.

The color RGB fusion circuit (52) also outputs signals to the image overlay controller (46) and a video input module (64) for reading the laser light pattern (62) projected onto the targeted anatomy at the surgical site (59) by the laser pattern projector (61). A processing module (65) processes the laser light pattern (62) and outputs a first video output signal (66) representative of the distance to the visible tissue at the surgical site (59). The data is provided to the image overlay controller (46). The processing module (65) also outputs a second video signal (68) representative of a three-dimensional rendered shape of the tissue or organ of the targeted anatomy at the surgical site.

The first and second video output signals (66, 68) include data representative of the position of the critical structure on a three-dimensional surface model, which is provided to an integration module (69). In combination with data from the video output processor (45) of the spectral control circuit (42), the integration module (69) may determine distance (d_{A}) (FIG. 1) to a buried critical structure (e.g., via triangularization algorithms (70)), and that distance (d_{A}) may be provided to the image overlay controller (46) via a video out processor (72). The foregoing conversion logic may encompass a conversion logic circuit (41), intermediate video monitors (74), and a camera (56)/laser pattern projector (61) positioned at surgical site (59).

Preoperative data (75) from a CT or MRI scan may be employed to register or align certain three-dimensional deformable tissue in various instances. Such preoperative data (75) may be provided to an integration module (69) and ultimately to the image overlay controller (46) so that such information may be overlaid with the views from the camera (47) and provided to video monitors (74). Registration of preoperative data is further described herein and in U.S. Pat. Pub. No. 2020/0015907, entitled “Integration of Imaging Data,” published Jan. 16, 2020, for example, which is incorporated by reference herein in its entirety.

Video monitors (74) may output the integrated/augmented views from the image overlay controller (46). On a first monitor (74a), the clinician may toggle between (A) a view in which a three-dimensional rendering of the visible tissue is depicted and (B) an augmented view in which one or more hidden critical structures are depicted over the three-dimensional rendering of the visible tissue. On a second monitor (74b), the clinician may toggle on distance measurements to one or more hidden critical structures and/or the surface of visible tissue, for example.

D. Exemplary Hyperspectral Identifying Signatures

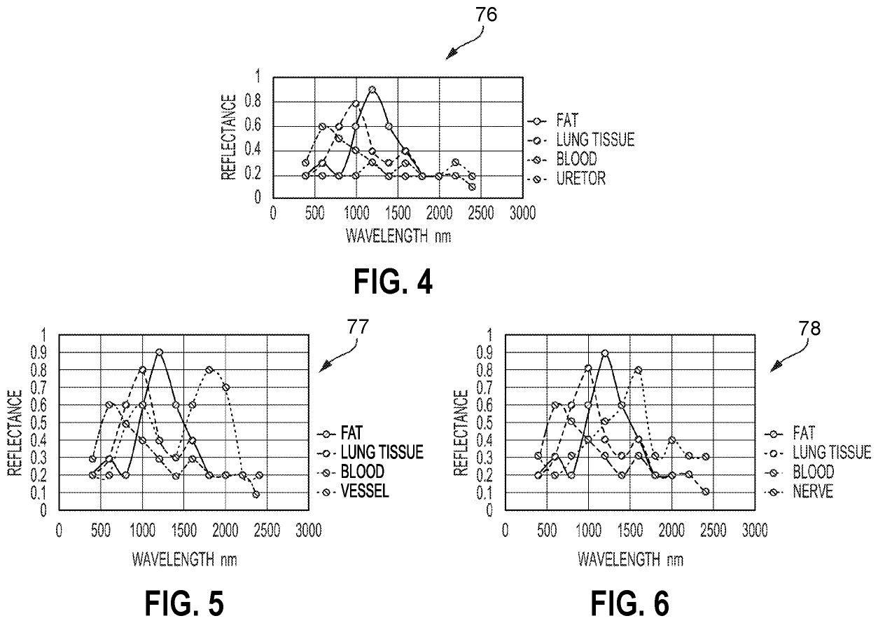

FIG. 4 depicts a graphical representation (76) of an illustrative ureter signature versus obscurants. The plots represent reflectance as a function of wavelength (nm) for wavelengths for fat, lung tissue, blood, and a ureter. FIG. 5 depicts a graphical representation (77) of an illustrative artery signature versus obscurants. The plots represent reflectance as a function of wavelength (nm) for fat, lung tissue, blood, and a vessel. FIG. 6 depicts a graphical representation (78) of an illustrative nerve signature versus obscurants. The plots represent reflectance as a function of wavelength (nm) for fat, lung tissue, blood, and a nerve.

In various instances, select wavelengths for spectral imaging may be identified and utilized based on the anticipated critical structures and/or obscurants at a surgical site (i.e., “selective spectral” imaging). By utilizing selective spectral imaging, the amount of time required to obtain the spectral image may be minimized such that the information may be obtained in real-time, or near real-time, and utilized intraoperatively. In various instances, the wavelengths may be selected by a clinician or by a control circuit based on input by the clinician. In certain instances, the wavelengths may be selected based on machine learning and/or big data accessible to the control circuit via a cloud, for example.

E. Exemplary Singular EMR Source Emitter Assembly

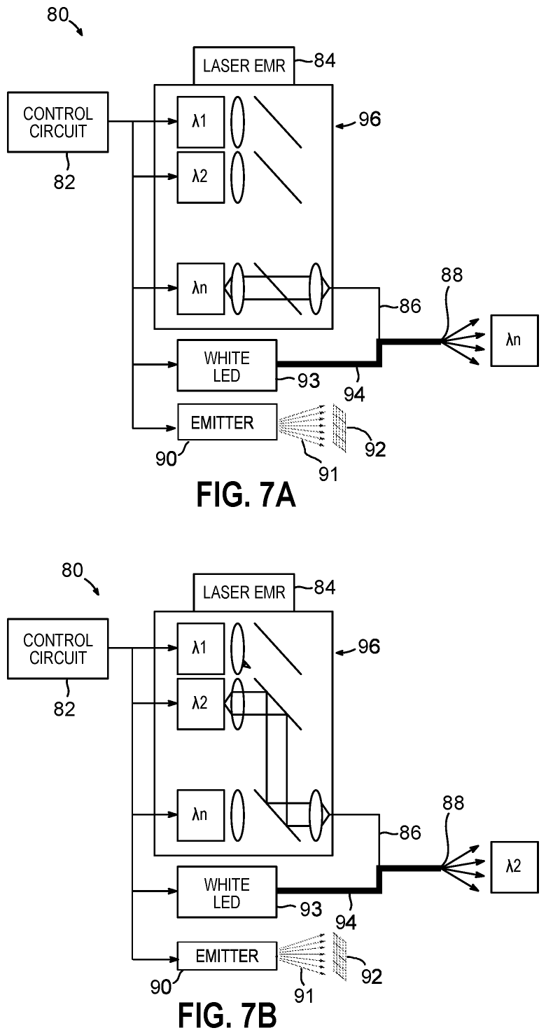

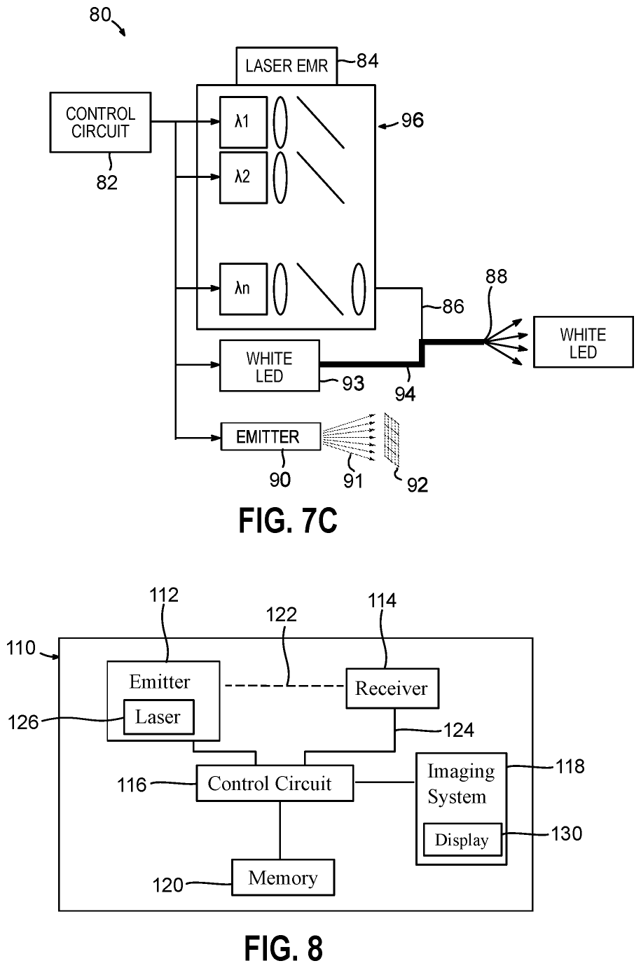

Referring now to FIGS. 7A-7C, in one aspect, a visualization system (10) includes a receiver assembly (e.g., positioned on a surgical device (16)), which may include a camera (47) including an image sensor (50) (FIG. 3), and an emitter assembly (80) (e.g., positioned on imaging device (17)), which may include an emitter (18) (FIG. 1) and/or a light engine (56) (FIG. 3). Further, a visualization system (10) may include a control circuit (82), which may include the control circuit (21) depicted in FIG. 2 and/or the spectral control circuit (42) depicted in FIG. 3, coupled to each of emitter assembly (80) and the receiver assembly. An emitter assembly (80) may be configured to emit EMR at a variety of wavelengths (e.g., in the visible spectrum and/or in the IR spectrum) and/or as structured light (i.e., EMR projected in a particular known pattern as described below). A control circuit (82) may include, for example, hardwired circuitry, programmable circuitry (e.g., a computer processor coupled to a memory or field programmable gate array), state machine circuitry, firmware storing instructions executed by programmable circuitry, and any combination thereof.

In one aspect, an emitter assembly (80) may be configured to emit visible light, IR, and/or structured light from a single EMR source (84). For example, FIGS. 7A-7C illustrate a diagram of the emitter assembly (80) in alternative states, in accordance with at least one aspect of the present disclosure. In this aspect, the emitter assembly (80) comprises a channel (86) connecting an EMR source (84) to a first emitter (88) configured to emit visible light (e.g., RGB), IR. The channel (86) may include, for example, a fiber optic cable. The EMR source (84) may include, for example, a light engine (56) (FIG. 3) including a plurality of light sources configured to selectively output light at respective wavelengths. In the example shown, the emitter assembly (80) comprises a white LED (93) connected to the first emitter (88) via another channel (94). A second emitter (90) is configured to emit structured light (91) in response to being supplied EMR of particular wavelengths from the EMR source (84). The second emitter (90) may include a filter configured to emit EMR from the EMR source (84) as structured light (91) to cause the emitter assembly (80) to project a predetermined pattern (92) onto the target site.

The depicted emitter assembly (80) further includes a wavelength selector assembly (96) configured to direct EMR emitted from the light sources of the EMR source (84) toward the first emitter (88). In the depicted aspect, the wavelength selector assembly (96) includes a plurality of deflectors and/or reflectors configured to transmit EMR from the light sources of the EMR source (84).

In one aspect, a control circuit (82) may be electrically coupled to each light source of the EMR source (84) such that it may control the light outputted therefrom via applying voltages or control signals thereto. The control circuit (82) may be configured to control the light sources of the EMR source (84) to direct EMR from the EMR source (84) to the first emitter (88) in response to, for example, user input and/or detected parameters (e.g., parameters associated with the surgical instrument or the surgical site). In one aspect, the control circuit (82) is coupled to the EMR source (84) such that it may control the wavelength of the EMR generated by the EMR source (84). In various aspects, the control circuit (82) may control the light sources of the EMR source (84) either independently or in tandem with each other.

In some aspects, the control circuit (82) may adjust the wavelength of the EMR generated by the EMR source (84) according to which light sources of the EMR source (84) are activated. In other words, the control circuit (82) may control the EMR source (84) so that it produces EMR at a particular wavelength or within a particular wavelength range. For example, in FIG. 7A, the control circuit (82) has applied control signals to the nth light source of the EMR source (84) to cause it to emit EMR at an nth wavelength (λn), and has applied control signals to the remaining light sources of the EMR source (84) to prevent them from emitting EMR at their respective wavelengths. Conversely, in FIG. 7B the control circuit (82) has applied control signals to the second light source of the EMR source (84) to cause it to emit EMR at a second wavelength (λ2), and has applied control signals to the remaining light sources of the EMR source (84) to prevent them from emitting EMR at their respective wavelengths. Furthermore, in FIG. 7C the control circuit (82) has applied control signals to the light sources of the EMR source (84) to prevent them from emitting EMR at their respective wavelengths, and has applied control signals to a white LED source to cause it to emit white light.

In addition to the foregoing, at least part of any one or more of the surgical visualization system (10) depicted in FIG. 1, the control system (20) depicted in FIG. 2, the control system (40) depicted in FIG. 3, and/or the emitter assembly (80) depicted in FIGS. 7A and 7B may be configured and operable in accordance with at least some of the teachings of U.S. Pat. Pub. No. 2020/0015925, entitled “Combination Emitter and Camera Assembly,” published Jan. 16, 2020, which is incorporated by reference above. In one aspect, a surgical visualization system (10) may be incorporated into a robotic system in accordance with at least some of such teachings.

II. Exemplary Automatic Surgical Scene Adaption

A. Overview

It is desirable for the surgical visualization system (10) to automatically recognize anatomical structures within an anatomical field of a patient with little or no user interaction. In some versions, automatically recognizing the anatomical structures within the anatomical field may result in minimal, if any, manual adjustments being made by the surgeon. Automatic adjustment of operating parameters of the surgical visualization system (10) may account for the extensive biological variability observed in vivo for complex surgical scenes. Additionally, it is beneficial to obtain images of the surgical scene that may not be already visible using standard approaches. In some versions, it may be beneficial to leverage previously obtained and/or current information to tune a surgical visualization system (10) to scan the various targets (e.g., desired anatomical structures) efficiently and effectively. As will be described in greater detail with reference to FIGS. 8-15, an exemplary surgical visualization system (110) may automatically adjust one or more hardware parameters and/or one or more software parameters to optimize detection performance of the surgical visualization system (110) within the surgical scene using exemplary methods (210, 310) or a combination of methods (210, 310).

B. Exemplary Surgical Visualization System

FIG. 8 schematically shows the surgical visualization system (110), which may be similar to the surgical visualization system (10) described above with reference to FIGS. 1-7B. The surgical visualization system (110) is configured for intraoperative identification of at least one anatomical structure. As will be described in greater detail below, the anatomical structure may include at least one critical structure (e.g., critical structures (11a-11b) of FIG. 1 and critical structures (512, 514, 516) of FIG. 13) and/or at least one background structure (e.g., background structure (518) of FIG. 13). Identification of anatomical structures may include guidance to or avoidance of the critical structures (11a-11b, 512, 514, 516) and/or the background structures (518) by a surgical device (e.g., surgical device (16)). In some versions, the surgical visualization system (110) may achieve a constant, or nearly constant, level of detection performance to sense the surroundings and automatically adjust/tune the surgical visualization system (110) while requesting limited user input.

With continued reference to FIG. 8, the surgical visualization system (110) includes an emitter (112), a receiver (114), a control circuit (116), an imaging system (118) and a memory (120). The emitter (112) is configured to emit a plurality of waveforms (122). While the waveform (122) is shown schematically as extending generally between the emitter (112) and the receiver (114), persons skilled in the art would appreciate that this schematically depicted waveform (122) would be reflected back to the receiver (114) from the anatomical field, rather than transmitted from the emitter (112) to the receiver (114) directly. In some versions, the emitter (112) includes at least one laser (126). The receiver (114) is configured to capture images (124) of waveforms (122). In other words, the receiver (114) captures a reflected portion of the waveform (122) that is reflected back to receiver (114) from the anatomical field and transmits this reflected portion of the waveform (122) to the control circuit (116) as the image (124). The emitter (112) and the receiver (114) are configured for multispectral imaging or hyperspectral imaging.

The control circuit (116) is in communication with the emitter (112), the receiver (114), the imaging system (118) and the memory (120). The control circuit (116) is configured to receive the images (124) from the receiver (114). The control circuit (116) is configured to automatically adjust at least one operating parameter (260) of the surgical visualization system (110) to aid in identification of at least one anatomical structure (e.g., critical structures (11a-11b, 512, 514, 516) and/or background structure (518)) in the anatomical field (510) (see FIG. 13) of the patient based on the image (124). The imaging system (118) may be similar to the imaging system (27), and the display (130) may be similar to the display (29) which are shown and described with reference to FIG. 2. The memory (120) may be similar to the memory (22) which is also shown and described with reference to FIG. 2. While not shown, the surgical visualization system (110) may include additional components (e.g., components relating to the control systems (20, 40) shown in FIGS. 2-3).

C. First Exemplary Method

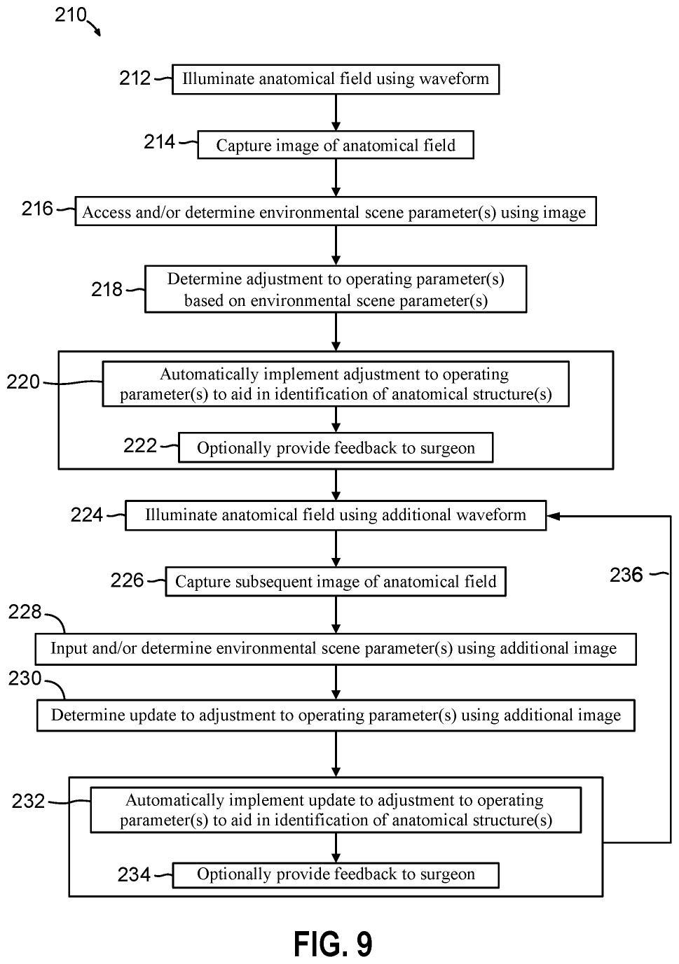

An exemplary method (210) of operating the surgical visualization system (110) is described with reference to FIG. 9. In some versions, an initial sensing prior to starting the surgery may assess the surgical scene for tissue signatures to adjust recipe selection for emitter (112) (e.g., laser (126)). At step (212), the method (210) includes illuminating the anatomical field (510) of the patient using the waveform (122) transmitted by the emitter (112). At step (214), the method (210) includes capturing the image (124) of the anatomical field (510) based on the waveform (122) using the receiver (114).

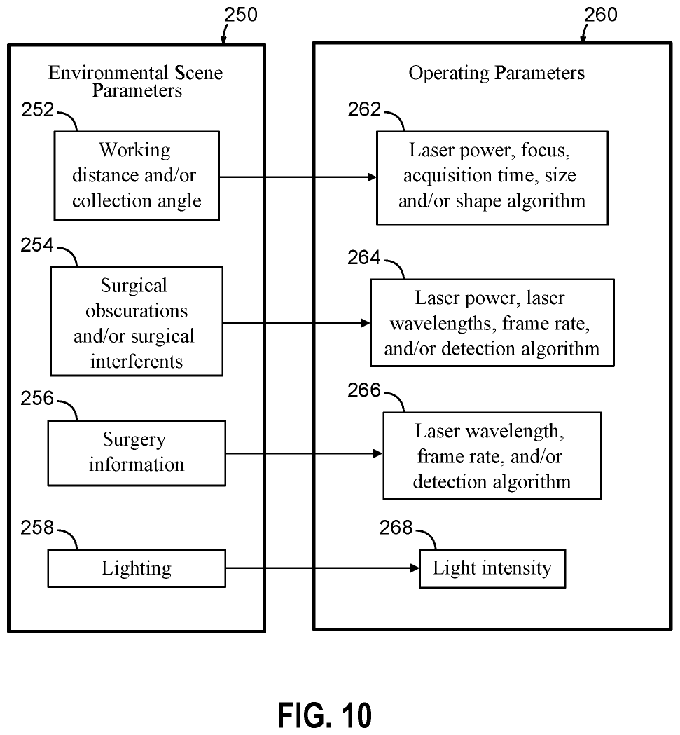

At step (216), the method (210) includes accessing or determining at least one environmental scene parameter (250) of the surgical visualization system (110) using the image (124). As shown in FIG. 10, a non-exclusive listing of environmental scene parameters (250) includes working distance and/or collection angle (252), surgical obscurations and/or surgical interferents (254), surgery information (256), and/or lighting (258), though other environmental scene parameters (250) be used either in addition to, or as alternatives to, those listed in FIG. 10. Moreover, multiple environmental scene parameters (250) may be used in combination with one another to aid the surgical visualization system (110) in identification of at least one anatomical structure (e.g., critical structures (11a-11b, 512, 514, 516) and/or background structure (518)). The critical structures (11a-11b, 512, 514, 516) may include one or more of an artery, a nerve, a vein, a common bile duct, a ureter, or a tumor, though the approaches disclosed herein may also be used to aid in identification of other types of critical structure as well. Accessing the surgery information (256) is further described with reference to the method (310) regarding obtaining/accessing surgery information specific to the patient (e.g., step (320)).

At step (218), the method (210) includes determining an adjustment to one or more operating parameters (260) of the surgical visualization system (110) based on at least one environmental scene parameter (250). This may be done, for example, by determining whether a detection algorithm is able to successfully identify a target structure. For instance, if the image of the anatomical field is oversaturated such that the detection algorithm is unable to operate, then operating parameters such as the intensity of light used for illumination may be decreased so as to reduce the oversaturation. Similarly, if a detection algorithm is able to identify a portion of an image as potentially depicting a target structure, but is not able to make a determination with a required level of confidence, then an adjustment to the operating parameters may be made to optimize the ability of the algorithm to detect and identify structures in that portion of the image (e.g., a camera used to capture images may be refocused on the specific portion, the intensity of lighting may be adjusted to provide maximum illumination without saturation in that portion even though this may result in oversaturation elsewhere in the image, etc.). Other approaches to determining an adjustment to operating parameters may also be implemented. For example, in some cases, rather than controlling the adjustment of operating parameters based on the operation of an algorithm used to identify critical structures, this determination may be based on optimizing parameters such as signal to noise ratio for the image, or modifying laser power based on the relationship between camera counts and some threshold value (discussed in more detail infra). Accordingly, the above description should be understood as being illustrative only, and should not be treated as implying limitations on potential manners in which step (218) may be performed. At step (220), the method (210) includes automatically implementing the adjustment to one or more operating parameters (260) to aid in identification of anatomical structures (e.g., critical structures (11a-11b, 512, 514, 516) and/or background structure (518)) in the anatomical field (510).

There are a variety of different of operating parameters (260) that may impact the surgical visualization system (110). The operating parameters (260) may include emitter power (e.g., laser power), emitter wavelength (e.g., laser wavelength), focus, acquisition time, size and/or shape algorithm, frame rate, detection recipe/algorithms, and light intensity, camera gain, camera binning, and multiple laser illumination. Additional operating parameters (260) that aid in identification of anatomical structures (e.g., critical structures (11a-11b, 512, 514, 516) and/or background structure (518)) are also envisioned. Some operating parameters (260) may impact surgical visualization system (110) more than other operating parameters (260). The number of adjustable operating parameters (260) may vary. For example, it may be beneficial to adjust a single operating parameter (260), a portion of the operating parameters (260), or each of the operating parameters (260) to optimize detection performance of surgical visualization system (110) based on the surgical scene (e.g., the anatomical field (510)) being viewed. Multiple recipes may be executed for a difficult target and a decision fusion/voting scheme may be utilized to increase confidence and reduce false negatives.

As used herein, a “recipe” is an algorithm with a combination of mathematical operations that operate on the rendered image. In some versions, with molecular chemical imaging (MCI), the images rendered may be as simple as the ratio of two wavelengths (i.e., a non-absorbing wavelength and an absorbing wavelength). The recipe may have various shape parameters and/or textual parameters. The recipe may include elements of machine learning as well. For example, if trying to locate a nerve that is linear, tubular, relatively long, and thin, the recipe searching for the nerve searches for long and thin tubular structures. A recipe example may modify a tube detection to optimize or enhance longer thinner structures versus shorter stouter structures. The recipe may leverage known preclinical shape and known textures to improve detection performance.

i. Environmental Scene Parameter Including Working Distance and/or Collection Angle

As shown in FIG. 10, when the environmental scene parameter (250) includes working distance (range to target) and/or collection angle (252), the corresponding operating parameters may include one or more of a power of the emitter (112), a focus, an acquisition time, a size algorithm, and/or a shape algorithm (262). Based on environmental scene parameters such as the working distance and/or collection angle (252), the surgical visualization system (110) may automatically adjust one or more of the power of the emitter (112), the focus, the acquisition time, the size algorithm, and/or the shape algorithm (262).

For example, regarding working distance, when moving closer to the surgical scene (i.e., decreasing the working distance), the laser power may be reduced, the laser pulse widths may be reduced, and/or camera sensitivity may be reduced by the surgical visualization system (110). Adjusting one of more of these may improve and optimize detection performance of surgical visualization system (110). Conversely, when moving away from the surgical scene (i.e., increasing working distance), the laser power may be increased, the laser pulse widths may be increased, and/or camera sensitivity may be increased by the surgical visualization system (110).

For example, based on the reflections from the surgical scene as a function of angle, reflections and pixel saturations may start to occur. As a result of reflections and pixel saturations, the surgical visualization system (110) may automatically adjust one or more of the power of the emitter (112), the focus, the acquisition time, the size algorithm, and/or the shape algorithm (262). When the emitter (112) includes the laser (126), the operating parameters (260) may include at least one of a power of the laser (126) or a wavelength of the laser (126).

By increasing focus, a reduced surgical scene is captured resulting in greater distinction and definition of the edge surfaces of the viewed anatomical structures. The edge surfaces may be used for identification of anatomical structures (e.g., critical structures (11a-11b, 512, 514, 516) and/or background structure (518)). By decreasing focus, an enlarged surgical scene is captured which may result in a greater percentage of the surgical structure(s) being captured through the image affecting the ability to determine the anatomical structures present. Generally, viewing a larger portion of the structure allows for greater confidence structure recognition. Contrast may be increased (using the same or different interval) using a feedback loop in subsequent iterations (see arrow (236) in FIG. 9) better define of the anatomical structure. Particularly, contrast may be increased (by increasing focus) to a second contrast level to assist in identification of the anatomical structure. Should the surgical visualization system (110) not establish the desired confidence using the second contrast level, the contrast may be increased (by increasing focus) to a third contrast level. The process is repeated until a desired confidence is established as to the anatomical structure.

Algorithms may be adjusted on the fly in a variety of manners. In some versions, algorithms may be adjusted on the fly based on range to target, algorithm parameters may be automatically tuned, for instance, object detection methods may benefit from knowing the number of pixels a certain target is expected to cover in the field of view. Particularly, automatic scene-based laser power adjustment may vary individual power levels at each wavelength based on working distance, target obscuration, ambient light levels, and/or background clutter. For example, when viewing the surgical scene, an initial assessment of signal contrast or signal to noise ratio (SNR) may be performed. Based on this initial assessment, individual laser wavelength pulse widths may be adjusted (through additional iterations (see arrow (236) of FIG. 9) to optimize that SNR. SNR may utilize the ratio of pixel intensities, producing an intensity map. This may ensure the desired level of saturation is implemented at the adjustment step(s) to enhance viewing of the surgical scene. In some instances, camera gain, integration time, laser power, and other parameters may be automatically adjusted to ensure minimal saturation or maximum detection performance (e.g., using SNR and/or the algorithm). This may be similar to auto white light exposure. Additional sensing technology (e.g., laser ranging, stereovision cameras, radar) to track the working distance and/or collection angle (252) for adjusting algorithm size and shape parameters are also envisioned.

ii. Environmental Scene Parameter Including Surgical Obscurations and/or Surgical Interferents

According to another example, when the environmental scene parameter (250) includes surgical obscurations and/or surgical interferents (254), a corresponding operating parameter may include one or more of power of the laser (126), wavelengths of the laser (126), a wavelength power of the emitter (112), a wavelength pulse width, gain of the receiver (112), pixel binning/grouping of the receiver (112), or frame rate (264). Based on the surgical obscurations and/or surgical interferents (254), the surgical visualization system (110) may automatically adjust at least one of a power of the emitter (112), a focus of the receiver (114), a wavelength power of the emitter (112), a wavelength pulse width, gain of the receiver (112), pixel binning/grouping of the receiver (112), a frame rate of the emitter (112), or a detection algorithm based on first parameter. For example, regarding altering pixel binning, if too little information is presented as determined at step (218), the surgical visualization system (110) may instruct the pixels may be combined at step (220). Alternatively, if too much information is presented as determined at step (218), the surgical visualization system (110) may instruct the pixels may be split into more pixels as implemented at step (220). As previously described, multiple adjustments to operating parameters (260) may be performed simultaneously.

A thick layer of fat and/or scar tissue may obscure the target structure underneath, with the alteration of different parameters (e.g., laser pulse width, laser power, etc.) producing different contrasts. The surgical visualization system (110) may auto tune in real time to determine the desired combinations. The surgical obscurations and/or surgical interferents (254) may include, for example fat, blood, collagen, peritoneum, etc. Other surgical obscurations and/or surgical interferents (254) are al so envisioned. Particularly, automatic adjustment of selected wavelengths for detection of a particular critical structure in the presence of different surgical obscurations and/or surgical interferents (254) or in the presence of different critical structures is envisioned. For example, a search for target A in the presence of fat may utilize different wavelengths than a search for target A in the presence of blood. Regarding emitter wavelengths, some obscurations have a wavelength (or a range of wavelengths) that the obscuration reflects particularly strongly as determined at step (218); these wavelength(s) may be shut off as implemented at step (220).

According to a specific example, when the emitter (112) includes the laser (126), the laser (126) may be automatically adjusted at step (220) to compensate for the surgical obscurations and/or surgical interferents (254). As the target is present in the detectable background, the detection algorithm (e.g., recipe) changes. Using a closed-loop feedback regulating circuit, the frequency of the laser (126) may be adjusted to select the spectrum at a particular time to detect the desired anatomical structures (e.g., critical structures (11a-11b, 512, 514, 516) and/or background structure (518)). Adjusting the power of the laser (126) based on each frame (e.g., image (124)) may allow for greater autonomous operation. Using a closed-loop feedback regulating circuit, the power of the laser (126) may be automatically adjusted on each frame.

The change from frame to frame is accounted for by taking into account the laser power setting. Known settings may include LaserPower λ1 and LaserPower λ2, which may change from one instance to another, but are known at the time of acquisition. In some instances, exposure may be assumed to be fixed. Regarding experimental values, camera counts (e.g., CountsMax λ1 and CountsMax λ2) may occur at each wavelength. Actual counts may be dependent on the material and/or the power of the laser (126). Wavelength processing may be based on the NormalizedSignal λ. In some versions, this may be performed by either filling the image sensor well capacity to the maximum allowable amount without saturating pixels or optimizing SNR as described above. To take into account the changing power of the laser (126), the normalization may be based on the power of the laser (126). For example:

NormalizedSignal λ1=CountsMax λ1/LaserPower λ1

NormalizedSignal λ2=CountsMax λ2/LaserPower λ2

and if:

CountsMax λ1<threshold

increase LaserPower λ1.

In some versions, LaserPower λ1 may be increased (using the same or different intervals) until the desired clarity of the surgical scene is obtained or the clarity of the surgical scene decreases. For example, if the clarity decreases, the desired LaserPower λ1 may be overshot (e.g., using simulated annealing) resulting in LaserPower λ1 being subsequently decreased, and with subsequent increases and decreases of LaserPower λ1 until the desired LaserPower λ1 is achieved. Thermal based laser tuning may fine tune specific wavelengths of the laser (126) that are fixed in hardware to allow for greater flexibility based on surgical scenes observed or to account for wavelength drifts over time. The optical path settings may be adjusted using liquid crystal tunable filter (LCTF) or acousto-optic tunable filters (AOTF) in addition to various MEMS type of mechanical adjustments that may be applied in real-time to the lens assembly components to vary focal length, aperture, etc. Since the power setting available as a knob for laser (126) may not be linear with the actual power of the laser (126) due to laser photon flux, a scaling factor may be incorporated to account for any laser photon flux.

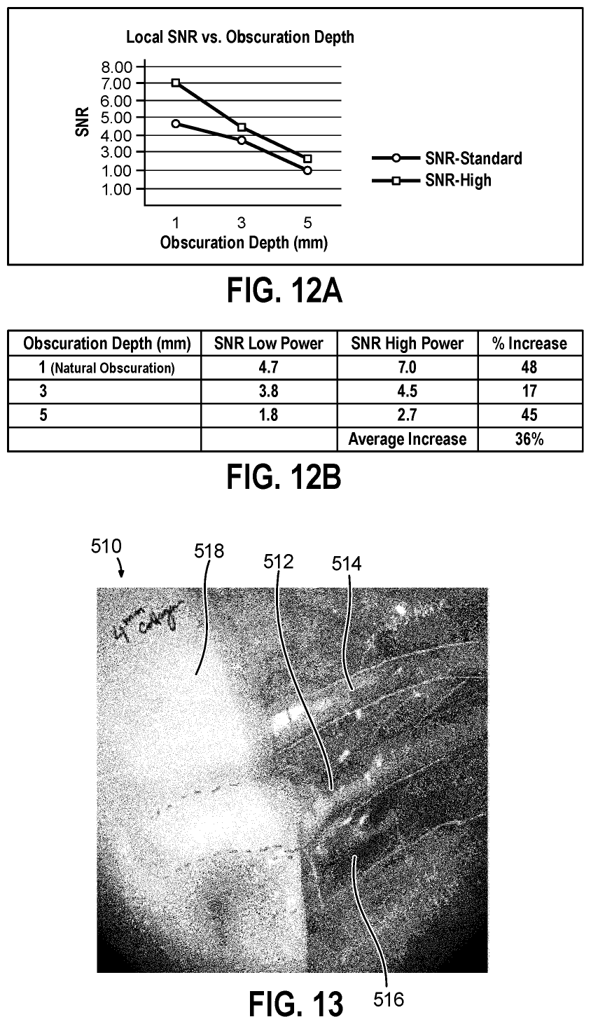

Frame rates may be adjusted for improved performance of the surgical visualization system (110). For difficult or buried targets, extra wavelengths or a combination of wavelengths may be deployed or acquisition time increased to trade latency for detection performance. Certain operating parameters (260) may be tuned/selected based on signal to noise ratio (SNR) levels observed in the surgical scene. For example, using preset values (e.g., based on the target based on expected reflectance and expected reaction at various wavelengths), an initial target search may be performed, with the target and background area being defined using the most confident detection pixels. Using these most confident detection pixels allows for tuning of operating parameters (260) to better understand the impact on the SNR. A (proportional-integral-derivative) PID type test of the control loop may be performed to execute this in real-time. A PID algorithm consists of three basic coefficients; proportional, integral and derivative which are varied to get an optimal response. Simulated annealing and/or gradient descent may also be utilized. High SNR levels may lower acquisition time to improve frame rate. Low SNR levels may increase acquisition time, which may improve detection rate at the cost of frame rate. Camera signal levels, noise levels, SNR may all be tracked for auto power of the laser (126), acquisition time adjustments, etc. Since extra wavelengths lower the frame rate, selectively adding and removing masks may improve the overall frame rate. FIG. 12A depicts a graph of an exemplary plot showing the relationship between standard and high signal to noise ratios (SNR) relative to obscuration depth, and FIG. 12B depicts a table of the relationship between SNR high (412) relative to a SNR standard (414) relative to obscuration depth of FIG. 12A. As generally shown, the higher the power level, the higher the SNR. Particularly, FIG. 12B shows there is about a 36% increase in a SNR high (412) relative to a SNR standard (414). Additionally, the SNR ratio decreases for both of SNR high (412) relative and SNR standard (414) as the obscuration depth increases.

iii. Environmental Scene Parameter Including Lighting

When the environmental scene parameter (250) includes lighting (258), a corresponding operating parameter may include light intensity (268). As a result, implementing the adjustment of step (220) includes automatically increasing the light intensity (268) to aid in the identification of anatomical structures (e.g., critical structures (11a-11b, 512, 514, 516) and/or background structure (518)) in the anatomical field (510). For example, when the surgical scene (e.g., anatomical field (510)) appears overly dim, the surgeon may increase the light intensity (268) of the light source, or this may be automatically increased in a manner similar to the automatic adjustments described for other parameters. In some versions, the brightness of the anatomical field (510) may be determined through the recognition of anatomical structures by the surgical visualization system (110) or through the use of a light sensor. It is desirable to have the increment between successive images (124) generally small, but larger when the anatomical field (510) is overly dark. It is also desirable avoid oversaturation. In some versions, oversaturation of the image may be avoided by lowering laser power, pulse widths, sensor integration time, or camera sensitivity/gain among other variables. The user may also slightly move the camera away from surgical scene. There are a variety of suitable auto exposure methods available that may be applied to laser power (light) settings instead of exposure based on the camera signal and the adjustment.

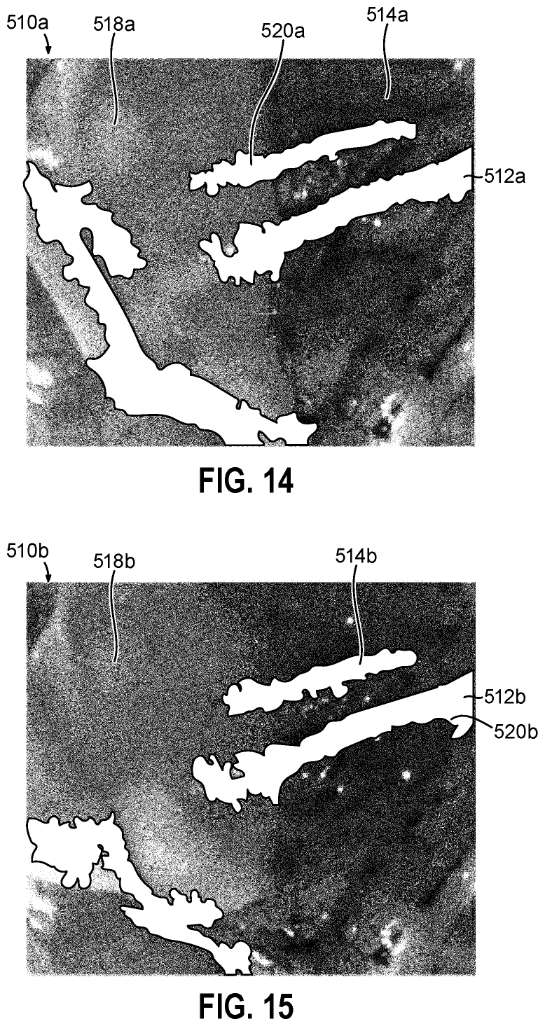

At step (222), the method (210) may optionally provide feedback to the user (e.g., the surgeon). Particularly, step (234) may include applying a mask (522a-522b) (see FIGS. 14-15) to one or more anatomical structures (e.g., critical structures (11a-11b, 512, 514, 516) and/or background structure (518)). The mask (522a-522b) may include a colored structure superimposed over the actual anatomical structure (512, 514, 516, 518) to aid the surgeon in identification of the anatomical structures. It is envisioned that different anatomical structures may be associated with predetermined colors that may be automatically set and/or adjustable by the user.

Steps (212, 214, 216, 218, 220, 222) may be generally repeated as steps (224, 226, 228, 230, 232, 234) as described below. Repeating these steps using a feedback loop may result in greater discernment of critical structures and/or background structures. At step (224), the method (210) includes illuminating the anatomical field (510) of the patient using another subsequent waveform (122) transmitted by the emitter (112) after automatically implementing the adjustment. The waveforms (122) having differing wavelengths allow for multispectral imaging or hyperspectral imaging. For example, the waveforms (122) may have a variety of different wavelengths that impact the ability of the surgical visualization system (110) recognize anatomical structures (e.g., critical structures (512a-512b, 514a-514b) and/or background structure (518a-518b)) as shown in FIGS. 14-15.

At step (226), the method (210) includes capturing a subsequent image (124) of the anatomical field (510) based on a subsequent waveform (122) using the receiver (114). At step (228), the method (210) includes determining an update to one or more environmental scene parameters (250) of the surgical visualization system (110) based on the subsequent image (124). At step (230), the method (210) includes determining a subsequent adjustment to at one operating parameter (260) of the surgical visualization system (110) based on the update to at least one environmental scene parameter (250). The subsequent adjustment is based on the anatomical field (510) being viewed in the subsequent image (124). In other words, the subsequent adjustment may refine the operating parameters (260) of the surgical visualization system (110) adjusted during the initial adjustment as well as optionally make additional adjustments to the operating parameters (260).

At step (232), the method (210) includes automatically implementing the subsequent adjustment to one or more operating parameters (260) to aid in identification of at least one anatomical structure in the anatomical field (510). At step (234), the method (210) may optionally provide feedback to the user (e.g., the surgeon). Step (234) may be similar to step (222) described above. It is envisioned that steps (224, 226, 228, 230, 232, 234) may be repeated (see arrow (236)) multiple times to further to aid in identification of at least one anatomical structure in the anatomical field (510). This feedback loop may enhance the identification of at least one anatomical structure in the anatomical field (510). For example, as the anatomical field changes by moving the surgical instrument, the operating parameters (260) may also change. The surgical visualization system (110) may repeat every nth frame to reassess the surgical scene or as indicated by the surgeon if moved to a new surgical location. While this occurs, the surgical visualization system (110) may be slow or in some instances temporarily unusable. As a result, in some versions, this initial sensing may utilize a separate tracking camera. Additional optics, robotics, and/or external sensors may also be included in the surgical visualization system (110). Additionally, it is envisioned that the method (210) may be performed using select emitted waveforms (122). For example, it is envisioned that only a portion (e.g., every third waveform, every fifth waveform, etc.) of the waveforms (122) may affect the operating parameters (260).

D. Second Exemplary Method Using Pre-operative Information

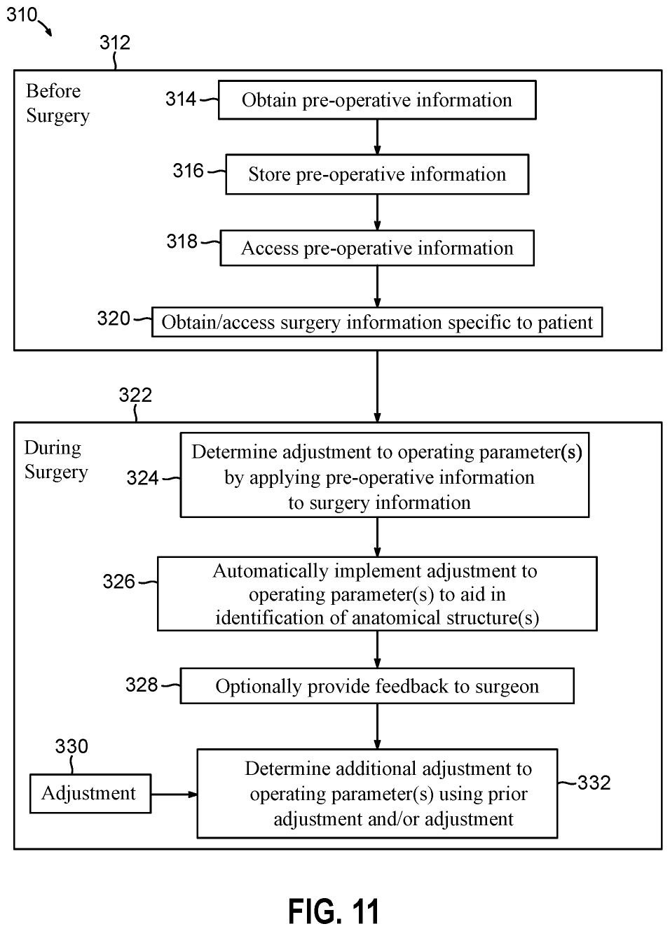

An exemplary method (310) of operating the surgical visualization system (110) using the surgical information (256) is described with reference to FIG. 11. As will be described below, it is envisioned that aspects of method (310) may be used in combination with aspects of the method (210) described above with reference to FIG. 9. As shown, the method (310) may include steps (314, 316, 318, 320) that may be performed before surgery (312) as well as steps (324, 326, 328, 330, 332) that may be performed during surgery (322).

At step (314), the method (310) includes obtaining pre-operative information. As used herein, the pre-operative information is information that is not specific to any particular patient that is subsequently used by the surgical visualization system (110) to aid in identification of at least one anatomical structure in the anatomical field (510). In some versions, pre-operative information may include information pertaining to one or more of a size of known critical structures, a shape of known critical structures, a size of known background structures, or a shape of known background structures. This pre-operative information may be used to train (e.g., through machine learning) the surgical visualization system (110) to better identify anatomical structures in the anatomical field (510).

At step (316), the method (310) includes storing the pre-operative information. It is also envisioned that this pre-operative information may be stored within or outside of the surgical visualization system (110) and accessed by the surgical visualization system (110) when desired. For example, the pre-operative information may be stored in the memory (120). The pre-operative information may be organized in a variety of manners, including being classified according to a particular surgical location and/or according to a particular surgical procedure. For example, FIG. 13 shows an exemplary anatomical field (510), where the displayed critical structures include a carotid artery (512), a vagus nerve (514), and a jugular vein (516). Background structures include collagen (518), which is illustrated as being about four millimeters thick. A variety of suitable critical structures and background structures are envisioned, and may be specifically based on the particular surgical location and/or particular surgical procedure the patient is to have performed. As a result, critical structures (11a-11b, 512, 514, 516) and/or background structures (520) may vary significantly. At step (318), the method (310) includes accessing pre-operative information from the memory (120). For example, this pre-operative information may be uploaded to and reside in a cloud and accessed by the surgical visualization system (110) when desired.

At step (320), the method (310) includes obtaining and/or accessing surgery information (256) of at least one of surgical location or surgical procedure that is specific to the patient. Accessing the surgery information (256) of at least one of surgical location or surgical procedure specific to the patient may help identify shapes and/or sizes of critical structures (11a-11b, 512, 514, 516) and/or background structures (520). In other words, having the surgical visualization system (110) recognize the surgical location and/or the surgical procedure to be performed allows the surgical visualization system (110) to use the previously obtained pre-operative information to identify anatomical structures based at least one of a size of known critical structures, a shape of known critical structures, a size of known background structures, or a shape of known background structures. For example, FIG. 13 depicts an exemplary anatomical field (510) that includes critical structures (512, 514, 516) and a background structure (518). As shown, the anatomical field (510) is marked up (i.e., annotated) by a medical professional to serve a “ground truth” of the size and positioning of the relevant anatomical structures (e.g., critical structures (512, 514, 516) and background structure (518)). This marked up anatomical field (510) may be used as and/or modified into pre-operative information in step (314) of method (310) to aid in identification of anatomical structures. The marked up anatomical field (510) may be used to check and validate the accuracy of images (510a-510b).

At step (324), the method (310) includes determining an adjustment to at least one operating parameter (260) by applying the pre-operative information to the surgery information (256). The operating parameters (260) may include a wavelength of emitter (112) (e.g., a wavelength of laser (126)), a wavelength power of the emitter (112), a wavelength pulse width, gain of the receiver (112) (e.g., camera gain), pixel binning/grouping of the receiver (112) (e.g., camera pixel binning/grouping), and/or a frame rate of the receiver (114) (e.g., camera frame rate) (266) using an algorithm of the surgical visualization system (110). The wavelength may be based on the particular wavelength being actively emitted by laser (126); however, employing multiple wavelengths simultaneously are also envisioned. Wavelength pulse width may refer to the amount of time the wavelength is actively emitted (i.e., duty cycle). The surgical location or surgical procedure generally determines the type and number of targets as well as the expected background target profile. For example, to detect a nerve in the neck compared to a nerve near the prostate, different backgrounds, different algorithms, and/or different hardware settings may be utilized. When the approximate shape and size of the target structure is known through pre-operative information, a detection algorithm may be used to identify structures having that approximate size and shape. For example, it may be beneficial to adjust a single operating parameter, a portion of the operating parameters, or each of the operating parameters to optimize detection performance of the surgical visualization system (110) based on the surgical scene (e.g., the anatomical field (510)) being viewed. Multiple recipes may be executed for a difficult target and a decision fusion/voting scheme may be utilized to increase confidence and reduce false negatives.

At step (326), the method (310) includes automatically implementing the adjustment to at least one operating parameter (260) to aid in identification of at least one critical structure in the anatomical field (510). The detection algorithm may include shape filters, recipes and other algorithm parameters may be optimized (i.e., shapes, sizes, temporal, thresholding, number of detections using prior information regarding the type of surgery and/or the region the surgical device is expected to be used on. The algorithms may be adjusted on the fly using statistical shape models of different types may be fit to the detected images based on the scene (ureter vs. common bile duct vs. tumor etc.).

At step (328), the method (310) includes providing real-time feedback to the user (e.g., the surgeon) on the display (130) to aid in identification of critical structures (11a-11b, 512, 514, 516) and/or background structures (520). The real-time feedback may include electronically displaying the mask (522a-b) on at least one critical structure (11a-11b, 512, 514, 516) and/or background structure (520) to aid in identification of critical structure (11a-11b, 512, 514, 516) and/or background structure (520). FIG. 14 depicts an image (510a) of the anatomical field (510) of FIG. 13, but with critical structures (512a, 514a) and/or background structure (518a) displayed using masks (520a) when image (510a) is captured at a first emitter power. FIG. 15 depicts an image (510b) of the anatomical field (510) of FIG. 13, but with critical structures (512b, 514b) and background structure (518b) using the masks (520b) when the image (510b) is captured at a second emitter power that is higher than the first emitter power. FIGS. 14-15 show automatic detection of the carotid artery at two distinct power levels of emitter (112). Using each of images (510a-510b) may provide for greater surgical identification than the use of images (510a-510b) by themselves. Critical structure (516) of FIG. 13 is not shown using a mask in FIGS. 14-15. Information may be captured for automated or minimal input adjustments. Regarding real-time feedback from the surgeon, if the surgeon notices the patient has a lot of fat obscuration, the surgeon may indicate that recipes should take fat into account to cause system to automatically adjust the operating parameters (260). Anti-material masks may be used to filter anatomical structures that are undesired during detection. For example, when target A and target B are both detected with wavelength 1 and wavelength 2, but Target B is also detected with wavelengths 3 and 4 which do not detect target A, wavelength 3 and 4 may be used to filter out target B.

At step (332), the method (310) includes determining an additional adjustment to one or more operating parameters (260) using the prior adjustment determined in step (324), and optionally an additional adjustment (330). For example, additional adjustment (330) may be based on steps (218, 230) of the method (210) described in detail with reference to FIG. 9. The surgery information (256) of the environmental scene parameter (250) may include at least one of a surgical location of the anatomical field (510) of the patient based on the surgical procedure pertaining to the anatomical field (510).

It is envisioned that step (332) may be repeated multiple times to further to aid in identification of at least one anatomical structure in the anatomical field (510). The subsequent adjustment is based on the anatomical field (510) being viewed in the subsequent image (124). In other words, the subsequent adjustments may refine the operating parameters (260) of the surgical visualization system (110) adjusted during the initial adjustment as well as optionally making additional or fewer adjustments to the operating parameters (260). For example, as the anatomical field changes by moving the surgical instrument, the operating parameters (260) may also change. Additionally, it is envisioned that the method (310) may be performed using select emitted waveforms (122). For example, it is envisioned that only a portion (e.g., every third, every fifth, etc.) of the waveforms (122) may affect the operating parameters (260).

III. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method of operating a surgical visualization system, the method comprising: (a) illuminating an anatomical field of a patient using a waveform transmitted by an emitter; (b) capturing an image of the anatomical field based on the waveform using a receiver, wherein the emitter and the receiver are configured for multispectral imaging or hyperspectral imaging; (c) determining an adjustment to at one operating parameter of the surgical visualization system based on at least one environmental scene parameter; and (d) automatically implementing the adjustment to the at least one operating parameter to aid in identification of at least one anatomical structure in the anatomical field.

### Example 2

The method of Example 1, further comprising determining the at least one environmental scene parameter of the surgical visualization system based on the image prior to determining the adjustment.

### Example 3

The method of Example 2, wherein: (a) the at least one environmental scene parameter comprises a first parameter from a group consisting of: (i) a working distance; and (ii) a collection angle; and (b) the act of automatically implementing the adjustment comprises automatically adjusting at least one of a power of the emitter, a focus, acquisition time, or a size and shape algorithm based on the first parameter.

### Example 4

The method of any one or more of Examples 2 through 3, wherein: (a) the at least one environmental scene parameter comprises a first parameter from a group consisting of: (i) a surgical obscuration, and (ii) a surgical interferent; and (b) the act of automatically implementing the adjustment comprises automatically adjusting at least one of a power of the emitter, a focus of the receiver, a frame rate of the emitter, or a detection algorithm based on the first parameter.

### Example 5

The method of any one or more of Examples 2 through 4, further comprising: (a) illuminating the anatomical field of the patient using a second waveform transmitted by the emitter after automatically implementing the adjustment; (b) capturing a second image of the anatomical field based on the second waveform using the receiver; (c) determining an update to the at least one environmental scene parameter of the surgical visualization system based on the second image; (d) determining a second adjustment to the at one operating parameter of the surgical visualization system based on the update to the at least one environmental scene parameter; and (e) automatically implementing the second adjustment to the at least one operating parameter to aid in identification of the at least one anatomical structure in the anatomical field.

### Example 6

The method of Example 5, wherein the second waveform has a different wavelength than the waveform.

### Example 7

The method of any one or more of the preceding Examples, wherein the at least one environmental scene parameter comprises a parameter from the group consisting of: (a) a surgical location of the anatomical field of the patient; and (b) a surgical procedure pertaining to the anatomical field of the patient.

### Example 8

The method of Example 7, wherein the method comprises: (a) accessing pre-operative information from a memory; (b) accessing surgery information of at least one of the surgical location or the surgical procedure specific to the patient; and (c) determining the adjustment to the at least one operating parameter applying the pre-operative information to the surgery information.

### Example 9

The method of Example 8, wherein the anatomical structure includes at least one of a critical structure or a background structure, wherein the pre-operative information comprises information from the group consisting of: (a) a size of known critical structures; (b) a shape of known critical structures, (c) a size of known background structures; and (d) a shape of known background structures.

### Example 10

The method of Example 9, wherein the at least one critical structure comprises a structure from the group consisting of: (a) an artery; (b) a nerve; (c) a vein; (c) a common bile duct; (e) a ureter; and (f) a tumor.

### Example 11

The method of any one or more of Examples 8 through 10, wherein the at least one operating parameter comprises a parameter from the group consisting of: (a) a wavelength of the emitter; (b) a wavelength power of the emitter; (c) a wavelength pulse width; (d) gain of the receiver; (e) pixel binning or grouping of the receiver; (f) a frame rate of the receiver; and (g) a detection algorithm of the surgical visualization system.

### Example 12

The method of any one or more of the preceding Examples, wherein the at least one environmental scene parameter includes lighting, wherein the operating parameter includes light intensity, wherein implementing the adjustment comprises automatically increasing an intensity of light to aid in the identification of the at least one anatomical structure in the anatomical field.

### Example 13

The method of any one or more of the preceding Examples, wherein the emitter includes a laser, wherein the at least one operating parameter comprises a parameter from the group consisting of: (a) a power of the laser and (b) a wavelength of the laser.

### Example 14

The method of any one or more of the preceding Examples, further comprising providing real-time feedback to a user on a display to aid in the identification of the at least one anatomical structure.

### Example 15

The method of Example 14, wherein the anatomical structure includes at least one critical structure, wherein the act of providing the real-time feedback comprises electronically displaying a mask on the at least one critical structure to aid in the identification of the at least one critical structure.

### Example 16

A method of operating a surgical visualization system, the method comprising: (a) illuminating an anatomical field of a patient using a waveform transmitted by an emitter; (b) capturing an image of the anatomical field based the waveform using a receiver, wherein the emitter and the receiver are configured for multispectral imaging or hyperspectral imaging; and (c) automatically adjusting at least one of a power of the emitter or a wavelength of the emitter to aid in identification of at least one anatomical structure in the anatomical field based on the image.

### Example 17

The method of Example 16, further comprising: (a) illuminating the anatomical field of the patient using a second waveform transmitted from the emitter after adjusting at least one of a power of the emitter or a wavelength of the emitter; (b) capturing a second image of the anatomical field based on the second waveform using the receiver; and (c) automatically adjusting at least one of a power of the emitter or a wavelength of the emitter based on the second image to aid in identification of the at least one anatomical structure in the anatomical field.

### Example 18

The method of any one or more of Examples 16 through 17, wherein the emitter includes a laser, wherein the at least one operating parameter includes at least one of a power of the laser or a wavelength of the laser.

### Example 19

A surgical visualization system comprising: (a) an emitter configured to emit a waveform; (b) a receiver configured to capture an image of the waveform; and (c) a control circuit in communication with at least the receiver and configured to receive the image from the receiver, wherein the control circuit is configured to automatically adjust at least one parameter of the surgical visualization system to aid in identification of at least one anatomical structure in an anatomical field of a patient based on the image.

### Example 20

The surgical visualization system of Example 19, wherein the emitter includes at a laser, wherein the at least one parameter includes at least one of a power of the laser or a wavelength of the laser.

IV. Miscellaneous

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

Furthermore, any one or more of the teachings herein may be combined with any one or more of the teachings disclosed in U.S. Pat. App. No. [Atty. Ref. END9342USNP1], entitled “Endoscope with Synthetic Aperture Multispectral Camera Array,” filed on even date herewith; U.S. Pat. App. No. [Atty. Ref. END9343USNP1], entitled “Endoscope with Source and Pixel Level Image Modulation for Multispectral Imaging,” filed on even date herewith; and/or U.S. Pat. App. No. [Atty. Ref. END9346USNP1], entitled “Stereoscopic Endoscope with Critical Structure Depth Estimation,” filed on even date herewith. The disclosure of each of these U.S. patent applications is incorporated by reference herein.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices described above may be designed to be disposed of after a single use, or they may be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, some versions of the device may be disassembled, and any number of the particular pieces or parts of the device may be selectively replaced or removed in any combination. Upon cleaning and/or replacement of particular parts, some versions of the device may be reassembled for subsequent use either at a reconditioning facility, or by a user immediately prior to a procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be sterilized before and/or after a procedure. In one sterilization technique, the device is placed in a closed and sealed container, such as a plastic or TYVEK bag. The container and device may then be placed in a field of radiation that may penetrate the container, such as gamma radiation, x-rays, or high-energy electrons. The radiation may kill bacteria on the device and in the container. The sterilized device may then be stored in the sterile container for later use. A device may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method of operating a surgical visualization system, the method comprising:
(a) illuminating an anatomical field of a patient using a waveform transmitted by an emitter;
(b) capturing an image of the anatomical field based on the waveform using a receiver, wherein the emitter and the receiver are configured for multispectral imaging or hyperspectral imaging;
(c) determining an adjustment to at one operating parameter of the surgical visualization system based on at least one environmental scene parameter; and
(d) automatically implementing the adjustment to the at least one operating parameter to aid in identification of at least one anatomical structure in the anatomical field.

2. The method of claim 1, further comprising determining the at least one environmental scene parameter of the surgical visualization system based on the image prior to determining the adjustment.

3. The method of claim 2, wherein:
(a) the at least one environmental scene parameter comprises a first parameter from a group consisting of:
(i) a working distance; and
(ii) a collection angle;
and
(b) the act of automatically implementing the adjustment comprises automatically adjusting at least one of a power of the emitter, a focus, acquisition time, or a size and shape algorithm based on the first parameter.

4. The method of claim 2, wherein:
(a) the at least one environmental scene parameter comprises a first parameter from a group consisting of:
(i) a surgical obscuration; and
(ii) a surgical interferent;
and
(b) the act of automatically implementing the adjustment comprises automatically adjusting at least one of a power of the emitter, a focus of the receiver, a frame rate of the emitter, or a detection algorithm based on the first parameter.

5. The method of claim 2, further comprising:
(a) illuminating the anatomical field of the patient using a second waveform transmitted by the emitter after automatically implementing the adjustment;
(b) capturing a second image of the anatomical field based on the second waveform using the receiver;
(c) determining an update to the at least one environmental scene parameter of the surgical visualization system based on the second image;
(d) determining a second adjustment to the at one operating parameter of the surgical visualization system based on the update to the at least one environmental scene parameter; and
(e) automatically implementing the second adjustment to the at least one operating parameter to aid in identification of the at least one anatomical structure in the anatomical field.

6. The method of claim 5, wherein the second waveform has a different wavelength than the waveform.

7. The method of claim 1, wherein the at least one environmental scene parameter comprises a parameter from the group consisting of:
(a) a surgical location of the anatomical field of the patient; and
(b) a surgical procedure pertaining to the anatomical field of the patient.

8. The method of claim 7, wherein the method comprises:
(a) accessing pre-operative information from a memory;
(b) accessing surgery information of at least one of the surgical location or the surgical procedure specific to the patient; and
(c) determining the adjustment to the at least one operating parameter applying the pre-operative information to the surgery information.

9. The method of claim 8, wherein the anatomical structure includes at least one of a critical structure or a background structure, wherein the pre-operative information comprises information from the group consisting of:
(a) a size of known critical structures;
(b) a shape of known critical structures;
(c) a size of known background structures; and
(d) a shape of known background structures.

10. The method of claim 9, wherein the at least one critical structure comprises a structure from the group consisting of:
(a) artery;
(b) a nerve;
(c) a vein;
(d) a common bile duct;
(e) a ureter; and
(f) a tumor.

11. The method of claim 8, wherein the at least one operating parameter comprises a parameter from the group consisting of:
(a) a wavelength of the emitter;
(b) a wavelength power of the emitter;
(c) a wavelength pulse width;
(d) gain of the receiver;
(e) pixel binning or grouping of the receiver;
(f) a frame rate of the receiver; and
(g) a detection algorithm of the surgical visualization system.

12. The method of claim 1, wherein the at least one environmental scene parameter includes lighting, wherein the operating parameter includes light intensity, wherein implementing the adjustment comprises automatically increasing an intensity of light to aid in the identification of the at least one anatomical structure in the anatomical field.

13. The method of claim 1, wherein the emitter includes a laser, wherein the at least one operating parameter comprises a parameter from the group consisting of:
(a) a power of the laser; and
(b) a wavelength of the laser.

14. The method of claim 1, further comprising providing real-time feedback to a user on a display to aid in the identification of the at least one anatomical structure.

15. The method of claim 14, wherein the anatomical structure includes at least one critical structure, wherein the act of providing the real-time feedback comprises electronically displaying a mask on the at least one critical structure to aid in the identification of the at least one critical structure.

16. A method of operating a surgical visualization system, the method comprising:
(a) illuminating an anatomical field of a patient using a waveform transmitted by an emitter;
(b) capturing an image of the anatomical field based the waveform using a receiver, wherein the emitter and the receiver are configured for multispectral imaging or hyperspectral imaging; and
(c) automatically adjusting at least one of a power of the emitter or a wavelength of the emitter to aid in identification of at least one anatomical structure in the anatomical field based on the image.

17. The method of claim 16, further comprising:
(a) illuminating the anatomical field of the patient using a second waveform transmitted from the emitter after adjusting at least one of a power of the emitter or a wavelength of the emitter;
(b) capturing a second image of the anatomical field based on the second waveform using the receiver; and
(c) automatically adjusting at least one of a power of the emitter or a wavelength of the emitter based on the second image to aid in identification of the at least one anatomical structure in the anatomical field.

18. The method of claim 17, wherein the emitter includes a laser, wherein the at least one operating parameter includes at least one of a power of the laser or a wavelength of the laser.

19. A surgical visualization system comprising:
(a) an emitter configured to emit a waveform;
(b) a receiver configured to capture an image of the waveform; and
(c) a control circuit in communication with at least the receiver and configured to receive the image from the receiver, wherein the control circuit is configured to automatically adjust at least one parameter of the surgical visualization system to aid in identification of at least one anatomical structure in an anatomical field of a patient based on the image.

20. The surgical visualization system of claim 19, wherein the emitter includes at least one laser, wherein the at least one parameter includes at least one of a power of the laser or a wavelength of the laser.
